(19) United States
(12) Patent Application Publication
(10) Pub. No.: US 2006/0004236 A1
(43) Pub. Date: Jan. 5, 2006

(54) Method of separating and converting hydrocarbon composites and polymer materials

(75) Inventor: James P. Barvincak, Ankeny, IA (US)

Correspondence Address: MCKEE, VOORHEES & SEASE, P.L.C., 801 GRAND AVENUE, SUITE 3200, DES MOINES, IA, 50309-2721, US

(73) Assignee: DREAMCO, INC., ANKENY, IA (US)

(21) Appl. No.: 11/174,776

(22) Filed: Jul. 5, 2005

Related U.S. Application Data
(63) Continuation-in-part of application No. 10/112,322, filed on Mar. 29, 2002, now abandoned.

(51) Int. Cl. C07C 1/00; C10G 1/00; C10G 1/10

(52) U.S. Cl. 585/240; 585/241; 208/400

(57) ABSTRACT

The process described herein presents a unique method of separating and converting polymers and hydrocarbon composites to light/medium weight hydrocarbons from polymer and hydrocarbon composites waste. The method involves the removal of moisture from the hydrocarbon composite and/or polymer waste followed by or in conjunction with a catalytic reaction which takes place at slight negative pressure in anaerobic conditions. The light/medium weight hydrocarbons are then recovered in the vapor phase. The vapors are condensed and separated by conventional techniques. Residual solids, substantially free of hydrocarbons and polymers may be furthered processed and recycled by conventional means. The conversion process has also been applied to natural occurring heavy and low grade hydrocarbon deposits such as oil and tar sands.

### PRIORITY CLAIM

This invention is a Continuation-in-Part application and claims priority to U.S. patent application Ser. No. 10/112,322, the disclosure of which is hereby incorporated by reference.

### FIELD OF THE INVENTION

This invention relates to a safe and efficient method of separating hydrocarbon composites and converting polymers to hydrocarbons from waste, waste products, and natural materials containing hydrocarbon composites and polymers into clean fuels and other useful products.

### BACKGROUND OF THE INVENTION

Wastes containing hydrocarbon composites and polymers are accumulating in large quantities in all highly industrialized countries. There is considerable concern in these countries for the impact of these wastes on the environment and human health. Within the petroleum industry alone there are numerous sources of hydrocarbon waste materials including: drilling by-products such as: the sand recovered in the mining of the oil sands; drilling by-products such as pump sand; processing waste such as tailing waste water, and refinery wastes to mention a few.

One of the largest oil sands (a mixture of sand, clay and bitumen) deposits in the world is in the Athabasca Basin in northeastern Alberta, Canada. Deposits are estimated to contain over 1.7 trillion barrels of bitumen and likely represent the largest accumulation of crude oil in the world (Quagraine et al., 2005). For each m³ of oil sand processed, about 3 m³ of water are required and this means about 4 m³ of fluid tailings are produced. The extraction tailings slurry consists mainly of solids (sand and clays), waster, dissolved organic and inorganic compounds, and un-recovered bitumen (MacKinnon, 1989; Mikula et al., 1996). The oil sands companies do not release any extraction wastes from their property leases, so that the process-affected waters and fluid tailings are contained on-site, primarily in large settling ponds. At the end of 1993, for example, the tailings ponds of both Syncrude and Suncor contain a total of approximately 300 million m³ of fine tailings. It is estimated that if the processes continue at the current rate, over 1 billion m³ of tailings pond water will require reclamation by 2025.

United States has oil sand deposits in California, Utah, Alabama, and Texas and elsewhere with reserves of 60 billion barrels of oil. The recovery cost has been prohibitive with current processes.

Oil shales are inorganic rocks that contain organic matter, mostly kerogen but some bitumen can also be present. Oil shales can contain greater than 50% organic matter by weight, or approximately 550 liters of oil per tonne of rock. Oil shales are often extracted using a retorting method wherein the shale is crushed and heated to approximately 500° C. with steam and the evolved liquid and gaseous products are collected. Retorting can also be performed in-situ by drilling two wells into an underground tunnel where explosions reduce the shale to rubble. In this method, steam is pumped into one well and the retorted oil is pumped up from the other. Environmental problems associated with these extraction methods include difficulty in disposing of the light fluffy ash by-product, as well as the necessity of using large quantities of water in order to process the shale.

Pump sand consists of the small amount of sand that is pumped out of the ground along with crude oil during oil exploration activities. After separation, the pump sand contains 5% to 10% crude oil plus 2% to 5% water. While this amount is approximately 1 kg pump sand per barrel of oil, as much as 200,000 barrels of crude oil/day is produced by major oil producers in one location. This results in a significant amount of pump sand. The cost of storing a tonne of pump sand typically ranges between $100 and $150/tonne. Thus, oil companies can spend up to $50,000 to $60,000 per day for storage of their pump sand which can impact ground water quality.

Another type of hydrocarbon drilling waste material is invert mud. When drilling for gas or oil, the drilling rigs pump a mixture of clay, water, and diesel fuel into the hole to keep it from collapsing. This mixture is recycled until the well is completed. The residual mud (invert mud) is difficult to reclaim. The invert mud is often stored in ponds, tanks, or salt mines to be removed at a later time. The cost of storing this invert mud ranges between $100/tonne and $150/tonne. Similar to pump sand, storage of hydrocarbon waste remains a significant source of pollution.

Oil refineries in the Houston area alone produce at least 240 tons/day of waste consisting of tank bottoms, crude oil spills/dirt, and tower wastes. At present, these refineries transport their waste to a disposal site in Louisiana at a cost of approximately $600/tonne. The annual waste disposal costs for these refineries are nearly $50 million per year.

Hydrocarbon wastes are produced in every industrialized country around the world where they are often stockpiled in legal and illegal landfills or remain on-site awaiting legislated waste management. Often regional or national regulations are lagging behind industry's needs. This delay in waste management detrimentally affects the environment. Some hydrocarbon waste materials are disposed of using inefficient and contaminating methods, while only a small percentage of these waste materials are reclaimed and reused.

Chambers (U.S. Pat. No. 4,235,676) and Xing (U.S. Pat. No. 6,133,491) summarize current processes of hydrocarbon extraction from organic waste. Processes involving the use of high temperatures and pressures have been known for many years. However, these known processes and apparatus have significant disadvantages. Chambers outlines these disadvantages including the loss of useful hydrocarbons through high temperatures and cross-chemical reactions of the reactants and products. Xing also expands on the difficulties associated with attempts to extract hydrocarbons in a vacuum and problems of repolymerization and condensation of some products at high temperatures. Chambers goes on to describe the formation of high molecular weight tars and hydrocarbons which reduces the yield of useable products.

Common current methods of waste treatment and disposal include incineration; thermal desorption; landfilling; solvent extraction; centrifugation; and, catalytic cracking. These methods are very expensive and often inefficient, resulting in air pollution. Furthermore, these methods produce minimal if any hydrocarbon recovery and result in a postponement of the waste disposal or separation of the chemical components. The invention presented herein presents an economically viable permanent solution.

Centrifugation as a method of hydrocarbon waste treatment involves the treatment of fuel oil tank bottoms, refining waste, pump sands, invert mud, and land/oil spills. Centrifugation involves the continuous separation of solid and liquid waste materials which are then discharged separately. The solids discharged from the centrifuge, however, have a residual liquid remaining. This resultant solid is referred to as the centrifuge cake. This centrifuge cake may contain up to 7% oil. In the past, this centrifuge cake has been left at the waste removal site or disposed in a landfill. In view of the environmental concerns of today, such disposal options are no longer acceptable. The current options for disposal of the centrifuge cake are storage include further disposal methods such as thermal desorption or incineration. All of these methods are very expensive and result in little or no recovery of usable hydrocarbon by-products.

Other hydrocarbon waste treatment processes involve cracking the polymers and hydrocarbon deposits. Cracking is a process whereby heavy hydrocarbon molecules are broken up into lighter molecules by means of heat and pressure (thermal cracking), and sometimes involve the use of catalysts (catalytic cracking). These cracking methods; however, are undertaken under extremely high temperatures and pressures and often involve the addition of hydrogen. These techniques are expensive, hazardous to operate, and are often inefficient in the complete recovery of the non-waste components.

With the present day concerns for the increasing cost of petroleum and the more readily recognized environmental impacts associated with hydrocarbon waste, there is an increasing interest in the efficient removal of useable hydrocarbons from hydrocarbon and polymer waste. In addition, with increasing petroleum consumption industry requires efficient techniques in recycling hydrocarbon and polymer waste.

Due to current air quality objectives and regulations, the current known methods of hydrocarbon and polymer waste treatment are limited by the required high temperatures, pressures, and the addition of harmful chemical additives. Also, the known techniques have been limited economically due to the high cost of operation. As a result, this invention presents a novel means of separating hydrocarbon composites and polymers from waste, waste products, and natural materials, such as oil sands and oil shale. This novel processing method allows for the separation of hydrocarbons in the form of medium-heavy oils, light oils, and gaseous hydrocarbons from petroleum waste products and natural hydrocarbon sources, leaving a clean residue in the form of residual solids, metals, minerals and ash which may be further processed into usable by-products. The recovered hydrocarbons can be further processed or burned for process heat or co-generation of steam and electricity.

Accordingly, it is a primary objective of the present invention to provide a novel method and means of processing and recycling hydrocarbon and/or polymer waste materials in addition to natural sources.

It is a further objective of the present invention to provide a novel method and means of processing and recycling hydrocarbons and/or polymers that is clean and efficient.

It is another objective of the present invention to provide an efficient method and means of processing and recycling hydrocarbons and/or polymers that results in more complete recovery of non-waste components than previous methods.

It is yet a further objective of the present invention to provide an economical method and means of processing and recycling hydrocarbons and/or polymers.

It is a further objective to provide a novel method and means of processing and recycling hydrocarbons and/or polymers at moderate temperatures to minimize hydrocarbon combustion of the recovered materials.

It is still a further objective of the present invention to provide a novel method and means of processing and recycling hydrocarbons and/or polymers resulting in re-useable and non-hazardous by-products.

It is a further objective of the present invention to provide a novel method and means of processing and recycling hydrocarbons and/or polymers that does not require the controlled disposal of waste by-products.

The method and means of accomplishing each of the above objectives as well as others will become apparent from the detailed description of the invention which follows hereafter.

### SUMMARY OF THE INVENTION

The present invention describes a method of processing and recycling hydrocarbons and/or polymers from waste materials and natural sources. The present method overcomes the difficulties in apparatus operation and the high costs associated with current methods of high temperature and pressure. As a result of the lower temperatures, slight negative pressure and anaerobic conditions fundamental to the present method, this novel means of processing and recycling hydrocarbon and polymer waste presents a cost effective, safe and efficient recovery technique required by industry.

The method presented herein involves the preconditioning of hydrocarbon and/or polymer waste by mechanical sizing, separation and dewatering as required. The preconditioned waste materials are then placed in a sealed heated vessel in the absence of oxygen, under slight negative pressures (from below 0 kPa to about −65 kPa). This step includes the reduction of the moisture content of the waste to less than 1% or less by weight.

Upon depletion of the moisture, the material is reacted in the presence of one or more selected catalysts at a temperature ranging between 100° C. and 1000° C. Appropriate catalysts include bauxite, bentonite, hydrosilicate, attapulgite, components of the montmorillonite clay family, silica-alumina and/or zeolite. The catalyst causes the polymer or hydrocarbons to separate or crack, and the temperature of the material to increase without the input of additional energy. During this reaction, hydrocarbons and converted polymers are vaporized and drawn off by an attached vacuum system. The processed material, substantially free of hydrocarbons and polymers is then allowed to cool. The heat recovered from the material may be further processed or recycled for use in the initial drying and heating step of this process or for the generation of steam or electricity. The amount of catalyst required is determined by the type and volume of waste. Typically, 1% to 10% catalyst by weight would be present in the reaction step. Agitation of the catalyst may be required in the case of specific input waste materials.

Separation of the hydrocarbon vapors is undertaken through the use of a cooling tower and/or distillation tower using conventional methods. Residual solids, substantially free of hydrocarbon and polymer waste are comprised primarily or non-hazardous materials and may be used in other processes or safely discarded. Water vapor recovered from the vapor phase and the drying of the solid waste is clarified by conventional means prior to re-use or disposal.

### BRIEF DESCRIPTION OF THE DRAWINGS

FIG. 1 is a flow chart showing a preferred hydrocarbon reclamation process of the invention.

FIG. 2 illustrates a preferred system of processing hydrocarbons in accordance with this invention.

FIG. 3 presents Cross Section A-A′ indicated in FIG. 2.

FIG. 4 illustrates the preferred feed systems for treatment of various types of raw materials in accordance with the processes of the invention.

### DETAILED DESCRIPTION OF THE PREFERRED EMBODIMENT

The present invention relates to the development of an efficient, safe, and clean method of separating hydrocarbon and polymer composites from waste, waste products, and natural materials. The procedure involves the placement of hydrocarbon composites and polymer-bearing materials along with a catalyst in a heated vessel depleted of most of its oxygen, under slight negative pressure, to create a reaction that allows the hydrocarbon and/or polymer components to be separated from the material in the form of gas or vapors.

The invention is useful for treating any waste or natural material that contains hydrocarbons or polymers. Examples of hydrocarbon and polymer waste products include, but are not limited to, pump sand, tailings waste water and sludge, refinery wastes, petroleum contaminated soil, tank bottoms, sludge, invert or drilling mud, used oil filters, naturally occurring oil-bearing soil, oil sands, plastics, oil shale, coal, oil absorbents, and creosote.

The input waste materials are first segregated and reduced in size if necessary. Excess moisture is removed, typically by centrifugation. Subsequent to preconditioning, the process involves further removal of moisture from the hydrocarbon and/or polymer-bearing materials. This is preferably accomplished by drying the materials by placing them in a vessel and heating to temperatures ranging between about 90° C. and 250° C., with about 100-150° C. being preferred, for a time period sufficient to reduce the moisture content of the materials, preferably to a moisture content less than 1% or less by weight. The most preferred temperature is about 110° C. In general, the higher the concentration of moisture in the material, the higher the preferred processing temperature. The drying time is adjusted in accordance with the temperature and the moisture content of the input materials.

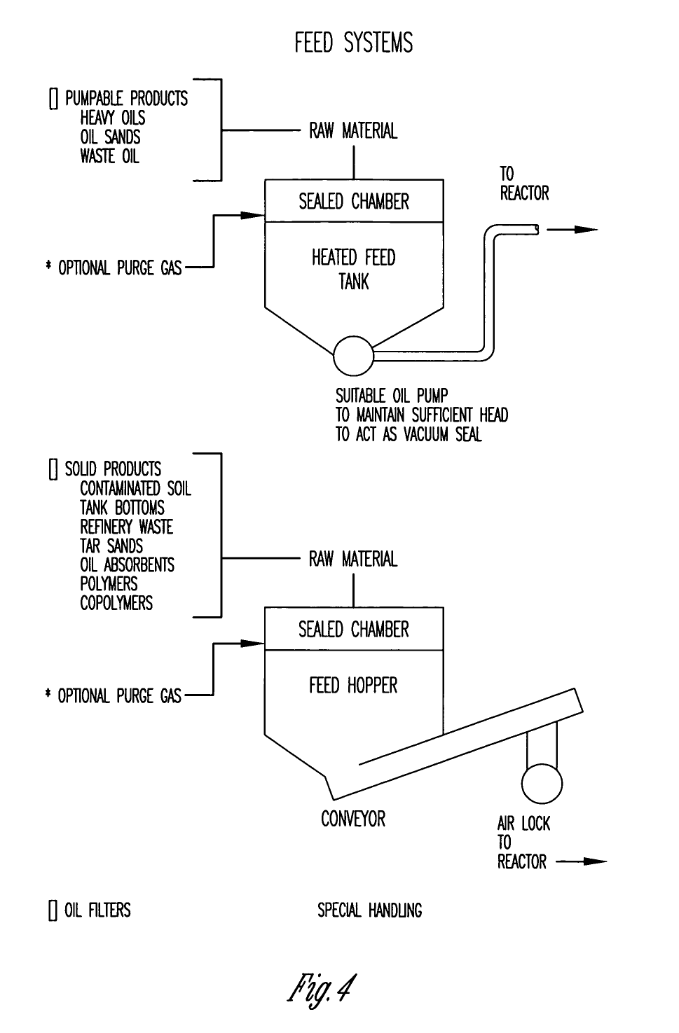

FIG. 4 illustrates preferred feed systems for the raw materials processed in the invention. As shown, the process used to handle the materials depends on the nature of the materials. For instance, pumpable products, such as heavy oils, oil sands, and waste oil are first placed in a sealed chamber that maintains the negative pressure and anaerobic conditions. Within the sealed chamber the materials are transferred to a heated feed tank, then fed through an oil pump that maintains sufficient head to act as a vacuum seal. Other raw materials, such as oil filters, require special handling. Specifically, oil filters need to be crushed and/or shredded before being fed to a special belt-style conveyor for processing because of the large volume of metal present.

The material is preferably preconditioned by means of sizing and excess water removal depending on the nature of the raw material. For instance, hydrocarbon contaminants, such as oil spills, soil, refinery waste, and storage tank residue are preferably screened, the material washed and allowed to settle, then separated via centrifugation. Natural oil sources such as heavy oils, oil sands, pump sands, and oil sands are preferably preheated to temperatures ranging between 90° C. and 250° C., with about 100-150° C. being preferred, the free water separated via centrifugation or evaporation, and the material ground and sized. Similarly, recyclables, such as waste oil, oil absorbents, and oil filters, are preheated to a similar temperature range. Manufactured products, such as plastics, are preferably shredded and sized prior to processing.

The moisture in the hydrocarbon and polymer-containing materials may also be removed using other conventional means known in the art including, but not limited to, centrifugation and air drying. Persons skilled in the art can readily appreciate such additional methods. It is generally preferred to centrifuge the materials prior to processing if they contain 10% or more moisture by weight.

During the drying process, moisture is preferably removed from the material as a vapor or gas. This is preferably accomplished by placing the material under a slightly negative pressure (i.e. a vacuum) ranging from less than 0 kPa to about −65 kPa, with less than 0 kPa to about −15 kPa being preferred, in order to encourage the flow of vapor. As used herein, the term “slight negative pressure” refers to any pressure that is less than neutral pressure but in a vacuum pressure equal to or less than about −65 kPa.

Moisture is removed from the waste material in a heated sealed unit which evaporates the moisture. This water vapor is removed by a vacuum generator which draws the vapor through a condenser where the vapor is liquefied.

The sealed vacuum chamber is critical to prevent combustion of light volatile hydrocarbons including hexane, gas condensates, acetone, ethanol, and other hydrocarbon by-products that have the same or a lower boiling point compared to water. As used herein, the term “anaerobic” refers to an atmosphere that contains insufficient oxygen to support an explosion during the processing of the hydrocarbons and/or polymers in accordance with this invention.

Once the moisture level of the hydrocarbon/polymer-containing material is reduced to an acceptable level, the material is reacted with at minimum of one catalyst at an elevated temperature which causes the hydrocarbon and/or polymer waste to “crack.” Examples of suitable catalysts for use in this invention include any catalysts typically used in refinery cracking methods including, but not limited to, catalysts containing aluminum hydrosilicate, bauxite, bentonite, attapulgite, and/or silica-alumina and zeolite. Such catalysts are well known in the art. Preferred catalysts include those from the montmorillonite clay family, with or without metals incorporated, such as nickel, molybdenum, cobalt, tungsten, iron, palladium, rhenium, tin, magnesium, and vanadium. The catalyst is added to the material in an amount of about 1% to 10% by weight of the material, with approximately 3% catalyst by weight being preferred. The material is reacted with the catalyst at temperatures ranging between about 100-1000° C., with about 110° C.-250° C. being preferred. During the reaction step, the catalyst and material may require agitation.

Mixing of the catalyst is not necessary. The waste material will react with the catalyst by placing the catalyst adjacent to or in close proximity with the material. The catalyst and material must be sufficiently close in proximity to allow the compounds to react. This distance will primarily depend on the temperature of the reaction and the type of catalyst used.

As noted above, the catalyst causes the hydrocarbon and/or polymer-containing material to crack and separate polymers and hydrocarbon composites from the material being processed. The cracking process causes a reduction of long-chain hydrocarbons, organic material, and polymers to convert to short-chained hydrocarbons with lower boiling points.

The catalytic cracking reaction typically increases the temperature of the waste material by about 25° C. to 200° C. (depending on the catalytic reaction and rate of removal of waste vapors) without the input of additional heat or energy into the process. As with the drying step, the catalytic reaction takes place at slight negative pressure ranging between less than 0 kPa to about −15 kPa. The catalytic reaction takes place in the same anaerobic sealed type chamber.

As stated previously, the above-referenced drying and catalytic reaction steps may take place simultaneously or independently depending on the material processed and the selected catalyst.

The catalytic reaction is preferably allowed to continue until all hydrocarbon and polymer vapors and gases are driven from the input waste material. The vapors or gases produced from the drying and catalytic reactions are separated from the waste material being processed, and may be condensed in a cooling or distillation tower as a liquid or liquefied gas. Some of the vapors produced range between C₁ and C₆, which remain in the gaseous state. This gaseous vapor is either collected and sold as surplus gas or burned as fuel to support the process. The recovered hydrocarbon oils and gases can be further processed into separate hydrocarbon fractions for use in the generation of steam and/or electricity.

The recovered material is allowed to cool, is preferably dried at a temperature ranging from about 90-250° C. (with about 100-150° C. being preferred), and then may be recycled in other industrial processes or safely discarded. The efficiency of the hydrocarbon removal is dependent upon composition of the input waste, the governing regulatory standards and/or the efficiency required by the waste owner.

The heat given off during the material's cooling process can be recovered and used to heat or dry the waste material at the beginning of this process or for the generation of steam or electricity. The substantially waste-free material often contains recoverable minerals, metals or residual solids suitable for construction fill.

Persons skilled in the art will readily understand that the processes described above may be performed in a one-step process, or in several steps. For instance, as already noted, the drying and catalytic steps may occur simultaneously, or take place in subsequent steps. While the drying and catalytic steps are occurring, the waste vapors may also be simultaneously removed and condensed.

In the alternative, the process of this invention may take place in several steps and in numerous chambers or containers in a factory or manufacturing process. For instance, the drying step may take place in a first chamber, the catalytic reaction step in a second chamber, and the separation step in a third chamber. Persons skilled in the art will also readily appreciate that the processes of this invention may be accomplished using a variety of equipment and techniques that are well known in the art, including conveyor belts, chambers, condensers, centrifuges, distillers, vacuum generators, etc. The specific equipment and processes used are not crucial to the removal of hydrocarbons.

The following examples are offered to illustrate but not limit the invention. Thus, they are presented with the understanding that various formulation modifications as well as method of delivery modifications may be made and still be within the spirit of the invention.

### EXAMPLE 1

### Preferred Hydrocarbon/Polymer Reclamation Process

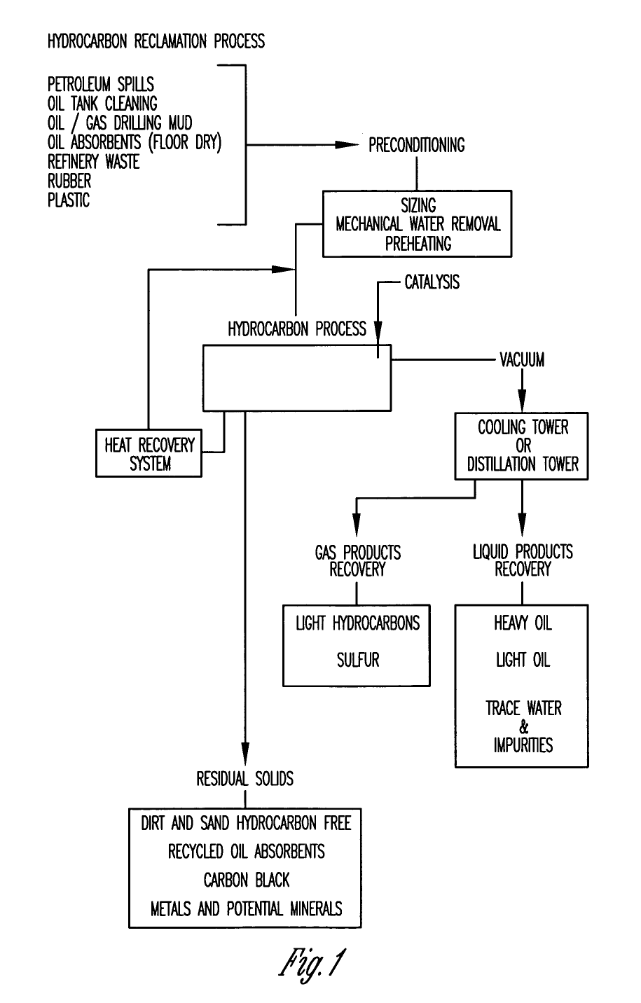

FIG. 1 illustrates a preferred hydrocarbon reclamation process in accordance with the present invention. Hydrocarbon and polymer waste materials, such as petroleum spills, oil tank cleaning, oil/gas drilling mud, oil absorbents, refinery waste, rubber, or plastic, as well as natural materials such as tar sand, oil sand, and heavy crude, are preconditioned by (a) reducing particle size; (b) removing excess water; and (c) heating.

The heated material next undergoes the catalytic reaction, and enters the “hydrocarbon process”. There, the waste gases and vapors are removed by vacuum and enter a cooling or distillation tower. The gaseous products are recovered in the form of light hydrocarbons, while the liquid products are recovered as heavy oils, light oils, trace water, and various impurities.

The material with the hydrocarbon/polymer removed is recovered and cooled. The heat given off by the material during the cooling process may be recovered and recycled for use in the heating and/or catalytic reactions. The residual solids, substantially free of hydrocarbon wastes and polymers, are comprised primarily of dirt, sand, recycled oil absorbents, carbon black (from rubber), metals, and minerals that may be recycled and used in other processes, or safely discarded.

### EXAMPLE 2

### Preferred Hydrocarbon/Polymer Reclamation Process

1. The mixture or compound of materials making up the hydrocarbon composites or polymer-bearing waste is reduced in size, conditioned, excess moisture removed by centrifugation, and fed into a machine line.

2. The material is fed into the first of three heated chambers equipped with internal conveyors and airlocks to support a normal negative operating pressure of less than 0 to about −15 kPa and anaerobic environment.

3. Once conveyed into the first chamber, moisture is removed by heating the mixture to a temperature of about 100-150° C. This elevated temperature drives the moisture from the mixture in the form of water vapor that is removed by the vacuum generator. This in turn draws the vapor through a condenser wherein the vapor is liquefied. The liquid is clarified through a centrifuge if necessary prior to disposal.

4. The dried material passes through an airlock and into a second heated chamber that supports a negative pressure of less than 0 to about −15 kPa. The chamber is equipped with a conveyor to move the material and provide agitation.

5. A refinery catalyst is added to the material in the second chamber and/or placed in a tray in close proximity to the material. The temperature of the material is raised to between 110-250° C., whereby the catalyst causes a reaction to take place with the hydrocarbon composites and polymers. This reaction, called cracking, raises the temperature of the mixture by about 25-200° C., depending on the rate that waste vapors are removed from the chamber and the type of hydrocarbon and/or polymer being removed. During the cracking reaction, hydrocarbon composites and converted polymers are vaporized and drawn off by the vacuum generator maintaining the negative pressure in the chamber.

6. The vapors are drawn through a condenser where they are condensed and collected. Some of the vapor is in the gas form and will not condense in the condenser. This gaseous vapor is either collected or burned as fuel to support the process. A small amount of the gas vapor or a small amount of inert gas can be introduced back into the chamber as purge gas to promote the removal of vapors being swept from the chamber.

7. Material conveyed through the second chamber is transferred into a third chamber equipped with a conveyor, heated to a temperature of about 100-250° C., and under a negative pressure of less than 0 to about −15 kPa. The catalytic reaction that began in the second chamber continues in the third chamber until all of the vapors have been driven and collected from the material. The vapors removed by the vacuum generator are treated the same as they were in the second chamber.

8. The processed material, free of hydrocarbon composites and polymers at the outlet end of the third chamber, is expelled through an airlock and allowed to cool. Some of the heat given off by the cooling mass of material can be used to warm material being prepared at the beginning of the process. Optional chambers may be added to handle a high volume of hydrocarbons or polymers if necessary. An additional chamber is used for a heat recovery system.

### EXAMPLE 3

### Preferred Equipment for Use in the Hydrocarbon/Polymer Reclamation Process

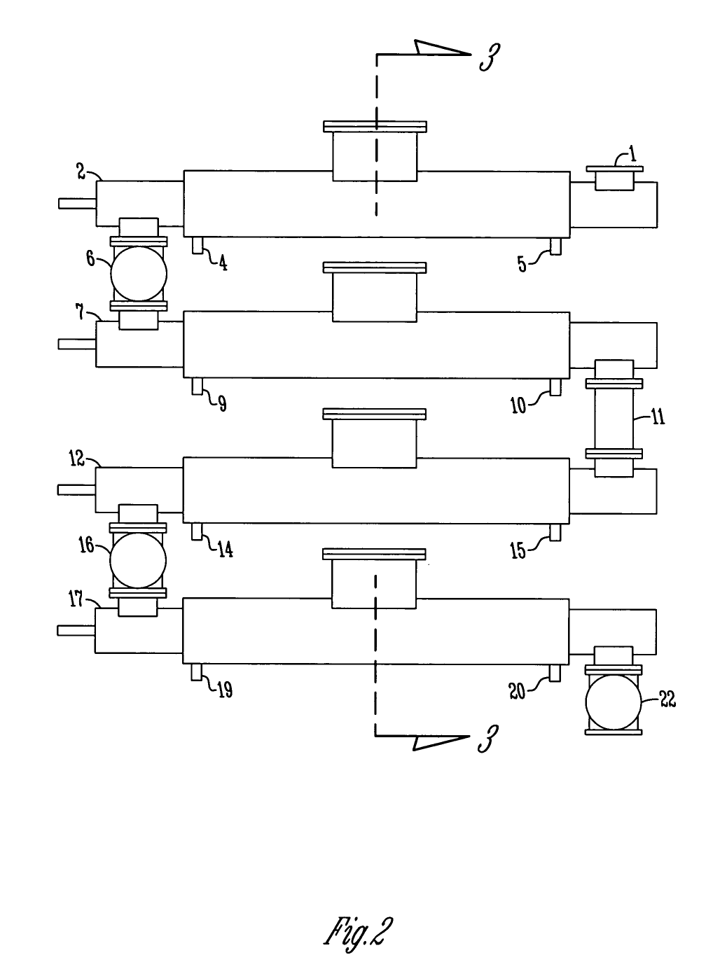

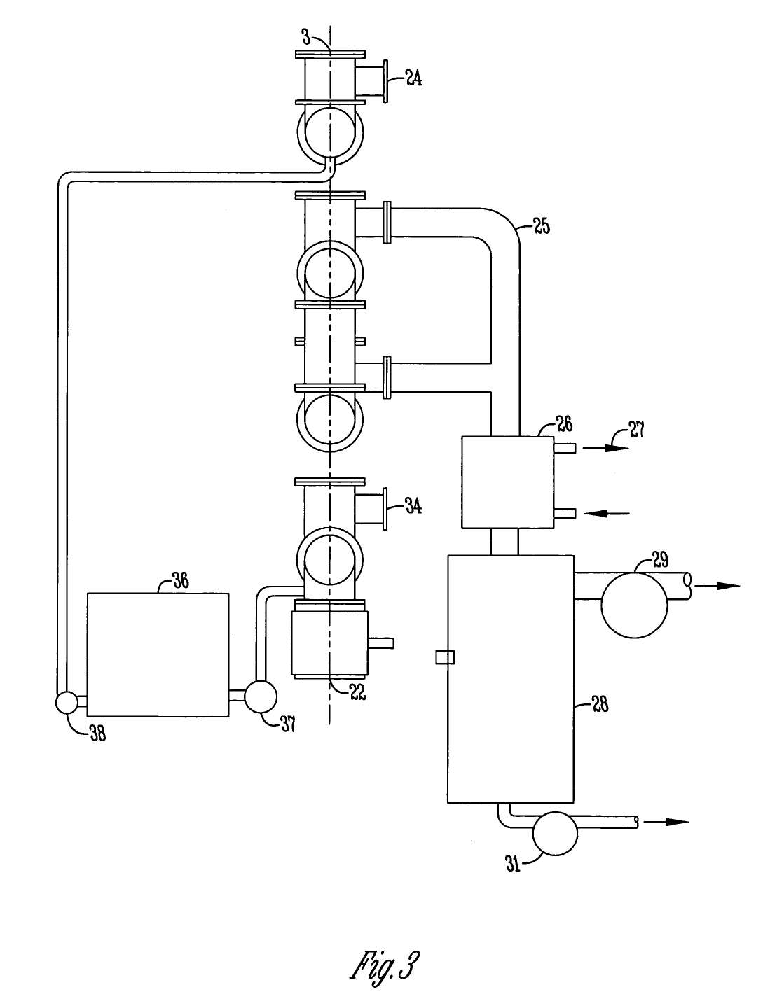

FIG. 2 and FIG. 3 illustrate preferred equipment for use in the hydrocarbon/polymer reclamation process of this invention.

Natural and/or waste material containing hydrocarbon and/or polymer is fed into the raw material inlet 1 where it proceeds to the first stage chamber, preheat, and water removal area 2. There, the material is heated using hot oil inlet 4 and dried to reduce its moisture content. Numeral 5 indicates the hot oil discharge area. Other hot oil inlets are indicated by numerals 9, 14, and 19, while other hot oil discharge areas are indicated by numerals 10, 15, and 20. Airlock 6 provides an atmosphere substantially free of oxygen. Other airlocks are indicated by numerals 16 and 22. Water vapor and potential light hydrocarbons produced during the drying stage is removed through a water vapor discharge 24.

The dried material next enters the second stage chamber 7 wherein it is reacted with a catalyst. Once the reaction is complete, the material is transferred through a transfer chute 11 to a third stage chamber 12 for providing additional heat and retention time to further separate the polymers/hydrocarbons from the material if necessary. Hydrocarbon/polymer vapors produced in the second and third stage chambers 7 and 12 are removed via hydrocarbon vapor discharge 25. The vapors enter a condenser 26 fed by cooling medium 27. The condensed hydrocarbons/polymer enter condensed oil chamber 28 and are removed for further processing via pump 31. Noncondensed hydrocarbons/polymers are removed from the system with a vacuum generator 29.

Heat from the earlier stages of the process may be recovered in a final stage 17 and recovered via pump 37. Excess heat is exhausted via hot air exhaust 34. The heat then enters hot oil recovery system 36 and is recycled via pump 38 to heat oil in the first stage chamber 2.

It should be appreciated that minor modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. A method of separating hydrocarbon composites and/or polymer composites from organic waste materials comprising: drying a material containing hydrocarbon and/or polymer; reacting the dried material with at least one catalyst for a time period sufficient to separate the hydrocarbon and/or polymer from the dried material; wherein the reacting step is performed at a slightly negative pressure.

2. The method of claim 1 whereby the reacting step is performed at a pressure ranging from less than 0 kPa to about −65 kPa.

3. The method of claim 2 whereby the reacting step is performed at a pressure ranging from less than 0 kPa to about −15 kPa.

4. The method of claim 1 whereby the reacting step takes place under anaerobic conditions.

5. The method of claim 1 whereby the drying step is performed at a slightly negative pressure.

6. The method of claim 1 whereby the drying step takes place at a temperature ranging from about 90-250° C.

7. The method of claim 6 whereby the drying step takes place at a temperature ranging from about 100-150° C.

8. The method of claim 1 wherein the material is dried until the moisture content of the material is ≦10% by weight.

9. The method of claim 8 wherein the material is dried to a moisture content of less than about 1% by weight.

10. The method of claim 1 wherein the drying step takes place under anaerobic conditions.

11. The method of claim 1 wherein the catalyst is selected from the group consisting of aluminum hydrosilicate, bauxite, bentonite, attapulgite, members of the montmorillonite clay family, silica-alumina, zeolite, and combinations of the same.

12. The method of claim 1 whereby the catalyst is present in a concentration ranging from about 1% to 10% by weight of the material.

13. The method of claim 1, wherein the dried material is reacted with the catalyst with agitation.

14. The method of claim 1 wherein the drying step and catalytic step are performed simultaneously.

15. The method of claim 1 whereby the reacting step takes place at a temperature ranging from about 100° C. to about 1000° C.

16. The method of claim 15 whereby the reacting step takes place at a temperature ranging from about 110° C. to about 250° C.

17. The method of claim 1 further including the step of segregating the separated hydrocarbon and/or polymer materials into vapor, gas, liquid and solid phases.

18. The method of 17 wherein the hydrocarbons and/or polymers are separated from the material through the use of a cooling tower or a distillation tower.

19. The method of claim 1 further including the step of drying the separated hydrocarbons and/or polymers.

20. The method of claim 19 further including the step of recovering heat used to dry the separated hydrocarbons and/or polymers.

21. The method of claim 20 further including the step of using the recovered heat in the step of drying the material.

22. The method of claim 1 wherein the drying and reacting steps are performed separately.

23. The method of claim 17 further including the step of burning the vapor as fuel in the drying and/or reacting steps.
